(19) United States
(12) Patent Application Publication
(10) Pub. No.: US 2024/0000357 A1
(43) Pub. Date: Jan. 4, 2024

(54) DEVICE AND METHOD FOR MEASURING A MUSCULAR STRENGTH OF A PATIENT

(71) Applicant: MYOCENE, Liège (BE)

(72) Inventors: Pierre RIGAUX, Liège (BE); Jean-Yves MIGNOLET, Momalle (BE)

(73) Assignee: MYOCENE, Liège (BE)

(21) Appl. No.: 18/251,936

(22) PCT Filed: Nov. 3, 2021
(86) PCT No.: PCT/EP2021/080477

§ 371 Date: May 5, 2023

Related U.S. Application Data
(63) Continuation-in-part of application No. 17/342,903, filed on Jun. 9, 2021.
Application No. 17/342,903 is a continuation-in-part of application No. 17/091,468, filed on Nov. 6, 2020.
Application No. 17/091,468 is a continuation-in-part of application No. 17/342,924, filed on Jun. 9, 2021.
Application No. 17/342,924 is a continuation-in-part of application No. 17/091,468, filed on Nov. 6, 2020.

(30) Foreign Application Priority Data
Nov. 6, 2020 (BE) 2020/5792

(51) Int. Cl. A61B 5/22; A61B 5/00

(52) U.S. Cl. CPC A61B 5/224; A61B 5/4884; A61B 5/6835; A61B 5/6828

(57) ABSTRACT

A device for measuring a muscular strength of a lower limb of a patient includes a seat for receiving the patient in a seated position and is suitable for being placed on a substantially horizontal mounting The device further includes an element mechanically coupled to the seat and supporting the lower limb so as to receive at least a portion of the patient's lower limb. A measurement instrument measures a force exerted by the lower limb at the element for supporting the lower limb and is configured to remain substantially immobile with respect to the support upon the application of a force by the patient's lower limb at the element for supporting the lower limb, by virtue of the patient's weight exerted on the seat.

### TECHNICAL FIELD

The present invention relates to a device and a method for measuring a muscular force of a patient.

### PRIOR ART

To carry out force measurements of a muscle, such as the quadriceps, several techniques are generally used.

A first technique used is an isokinetic apparatus, in which the patient is placed on a seat and strapped to it. The lower limb is attached to an articulated arm connected to a motor and a dynamometer which are connected to the chair. The movement of the patient is restricted so that it proceeds at a constant speed. A second technique, referred to as isotonic, consists in using heavy loads that the subject must displace. Similar to a weight bench, the patient is placed on a seat or a bench equipped with an articulated arm, connected either directly or by means of pulleys, to loads (weights, cast iron plates, etc.). The disadvantage of these techniques is that the apparatus used to implement those techniques is heavy and/or bulky, and provided to be permanently installed in a room dedicated to their use. A patient wishing to take a measurement must therefore go to a particular place.

A third technique allow to measure only the isometric force using a hand dynamometer. An operator separate from the patient holds the dynamometer in his hand by means of a strap and exerts a force opposite to that of the patient. In some cases the dynamometer is attached to the leg of a table via straps. This technique is impractical to use and the measurements obtained are not very accurate. When the operator holds the dynamometer, the isometric aspect is not guaranteed, as it is dependent on the movement of the patient and the pressure exerted by the operator. The measurement is approximate. In addition, the operator must be able to exert a force greater than that of the patient, which is rarely the case when measuring the maximum isometric force of the quadriceps of an experienced athlete. This technique is therefore rather limited to the rehabilitation. When the dynamometer is attached to a table, the positioning by means of straps is usually difficult, which also results in a very inaccurate and unreproducible measurement.

The document JP 2019-180555 A discloses a device for measuring the muscular force of the lower limbs. The device comprises a frame, an arm with one end attached to the frame and a sitting portion mounted on the frame, the sitting portion comprising a sitting surface and a backrest. A cushion is attached to the frame in front of the sitting portion so as to support the back of a knee of a subject seated on the sitting portion; a “leg” cushion portion is attached to the arm at a position corresponding to a portion of the leg under the knee of the patient when seated on the sitting portion. The device also comprises an instrument for measuring at least one muscular force exerted by pushing down (or up) the “leg” cushion portion with the back (or front) surface of the leg of the patient when seated on the sitting portion, with the “knee” cushion portion serving as a support point.

The disadvantage of the device of this document is that its structure is complex and heavy. The device is therefore not easy to transport.

The document U.S. Pat. No. 9,114,255 B1 discloses a brace for rehabilitating or exercising the knee joint of a subject. The brace comprises a narrow, elongated platform for accommodating an upper portion of a lower limb of a subject, coupled to a frame extending inferiorly from the platform and holding a transverse element, having a log-like shape, intended to be positioned in front of the tibia of the subject. The brace comprises a sensor in the frame at the level of the platform to detect an indirect force representative of a force exerted by the lower limb during a rehabilitation or an exercise.

This brace does not allow for accurate measurements of the force that is actually exerted by the lower limb, and this in particular as it does not provide a good stability during use.

There is therefore a need for a device for measuring a muscular force of a patient that is simpler and more convenient to transport while providing accurate measurements.

### DISCLOSURE OF THE INVENTION

For this purpose, the invention proposes a device for measuring a muscular force of a lower limb of a patient comprising a sitting for receiving the patient in a seated position and adapted to be placed on a substantially horizontal support, a support element for supporting a lower limb in order to receive at least a portion of the lower limb of the patient, mechanically coupled to the sitting, and a measurement instrument for measuring (preferably directly) a force exerted by the lower limb at the level of the support element for supporting the lower limb, the measurement device being configured such that it remains substantially immobile relative to the support upon the application of a force by the lower limb of the patient at the level of the support element for supporting the lower limb, due to the weight of the patient being exerted at the level of the sitting.

The instrument may be attached (directly, without an intermediary) to a part arranged to be aligned with the direction of the force exerted by the lower limb at the level of the support element for supporting the lower limb (in a (or in) configuration of use of the device). In other words, both equivalently and substitutably, the instrument can be attached (directly, without an intermediary) to a part aligned with the direction of the force, in a (or in) configuration of use of the device. Typically, the instrument may be intended to be aligned with the direction of the force, in one configuration of use of the device. Preferably, the measurement instrument is arranged in front of or behind the lower limb, more preferably at least partly aligned with a support point of the force along the direction of the force, so that it works in traction or compression when the device is in use.

These characteristics of the preceding paragraph concerning the positioning of the instrument may be substituted according to a preferred embodiment of the invention by the fact that the measurement instrument is a same part with the support element (in which case, the part to which the instrument is attached is none other than the support element).

In general, the instrument of the device is preferably arranged to allow a direct (rather than indirect) measurement of the force exerted by the lower limb at the level of the support element for supporting the lower limb.

The term “aligned” used above (and in the context of this document) is typically to be interpreted as the fact that the part (and/or the instrument) extends along (and preferably partially or totally symmetrically around) the direction of the force (considered as a physical vector with its support point). Preferably, a distance between the part (and/or the instrument) and the support element is less than 10 cm.

In the context of this document, as will be understood by a person skilled in the art, “the weight of the patient” generally refers to the entire weight of the patient. Since the sitting allows to receive the patient in a seated position and the weight of the patient is exerted at the level of the sitting, the device is particularly stable during its use, even when the force exerted at the level of the support element is great (for example, during an electro-stimulation contraction of a quadriceps muscle).

Optionally, the sitting is a tray extending continuously by at least 40 centimetres along two perpendicular axes. Preferably, the tray is rectangular in shape and has sides between 40 and 70 cm in length.

Optionally, the device comprises at least one arm mechanically coupling the support element for supporting the lower limb to the sitting.

Optionally, the angle between the arm or the arms and the sitting can be adjusted in one configuration of the device.

Optionally, the arm or the arms are folded against the sitting in a transport configuration of the device.

Optionally, the device comprises a single support element for supporting the lower limb, adjustable on the arm or the arms to adapt to different lower limbs.

Optionally, the arm or the arms offset the element out of the plane of the sitting, under the sitting, when the device is in use.

Optionally, the arm or the arms are movable in translation laterally with respect to the patient in the seated position. Similarly and optionally, the arm or the arms are movable in translation laterally in relation to the sitting.

Optionally, the measurement instrument is a strain gauge or a mechanical dynamometer.

Optionally, the measurement instrument can be adapted to work in compression or in traction.

Optionally, the device further comprises members for a removable positioning of the sitting on the support.

Optionally, the positioning members are height-adjustable.

Optionally, the device is configured such that the force exerted by the lower limb at the level of the support element for supporting the lower limb is substantially parallel to the sitting, in a use configuration of the device.

Optionally, the device is configured so that the support element for supporting the lower limb is able to receive a portion of the lower limb below the knee of the patient.

Optionally, the support element comprises a bight conformed to adapt to the portion of the lower limb resting against the element and/or comprises a strap for immobilising the lower limb against the support element. Optionally and similarly, the support element is such a bight comprising a rounded portion configured to match the curvature of the portion of the lower limb and laterally immobilise the lower limb, in a use configuration of the device.

Optionally, the muscle whose force is measured is a quadriceps.

Optionally, the support is a table.

Similarly, the invention also proposes a device for measuring a muscular force of a lower limb of a patient comprising:
a sitting for receiving the patient in a seated position and capable of being placed on a substantially horizontal support,
a support element for supporting the lower limb in order to receive at least a portion of the lower limb of the patient, mechanically coupled to the sitting by a mechanical arm or a mechanical frame,
a measurement instrument for measuring a force exerted by the lower limb at the level of the support element for supporting the lower limb,
the mechanical arm or the mechanical frame comprising a member for connecting to the instrument at the level of the support element,
the measurement device being configured such that it remains substantially immobile relative to the support when a force is applied by the lower limb of the patient at the level of the support element for supporting the lower limb, due to the weight of the patient being exerted at the level of the sitting.

This is an embodiment of the device as presented more generally in the first paragraph of the disclosure of the invention, so they share the same advantages. The fact that the instrument is linked to the mechanical arm or to the mechanical frame at the level of the support element means that the instrument is necessarily arranged at the level of the support element, and therefore as close as possible to the point where the force is exerted, typically aligned with the force, which allows direct measurements of the force, which are therefore more accurate.

Advantageously, the structure of the device is then very simple and light. The arm or the frame may have a simple shape, for example a projected “I”, “L”, “T”, “U”, “S” or “Z” shape in at least one plane orthogonal to the sitting, and preferably comprising at least one top end coupled (or attached) to the sitting, and at least one bottom end coupled (or attached) to the support element.

In particular, the case where a mechanical frame couples the sitting with the support element is a special case of “the arm or the arms” mentioned above.

In these respects, the foregoing embodiments and options, and their respective advantages, extend mutatis mutandis to the device comprising a mechanical arm or frame as described above.

The invention also relates to a method for measuring a muscular force of a lower limb of a patient, comprising the steps of providing the above described device, placing the sitting on a substantially horizontal support, positioning the patient seated on the sitting, positioning a lower limb of the patient whose force is to be measured in the support element for supporting the lower limb, taking force measurements of the lower limb at the level of the support element for supporting the lower limb.

Optionally, the patient is positioned so that his foot is off the ground.

Optionally, the entirety of a thigh of the patient rests on the sitting and the back of his knee is in contact with an edge of the sitting.

Optionally, the method further comprises a step of positioning the support element for supporting the lower limb opposite the lower limb whose muscular force is to be measured.

Optionally, the method further comprises adjusting the angle between the sitting and at least one arm mechanically coupling the support element for supporting the lower limb to the sitting and/or comprises adjusting the translation of the at least one arm laterally with respect to the patient seated on the sitting.

Optionally, the support is a table.

Optionally, the method is for measuring a muscular fatigue of a quadriceps or hamstring of a patient.

Optionally, the method further comprises an electro-stimulation step generating a force exerted by the lower limb at the level of the support element for supporting the lower limb.

Optionally, the method further comprises the steps of electro-stimulating a muscle of the lower limb, for example a quadriceps or a hamstring, at different frequencies, taking force measurements of the lower limb at the level of the support element for supporting the lower limb in response to the above-mentioned electro-stimulations, determining a muscular fatigue based on the force measurements of the lower limb taken in response to the above-mentioned electro-stimulations.

Optionally, the different frequencies comprise a first frequency and a second frequency, differing by at least 10% from each other, and the force measurements comprise a first force measurement of the lower limb at the level of the support element for supporting the lower limb in response to the electro-stimulation of the muscle at the first frequency, and a second force measurement of the lower limb at the level of the support element for supporting the lower limb in response to the electro-stimulation of the muscle at the second frequency.

Optionally, the determination of the muscular fatigue comprises a calculation of the ratio between the first and the second force measurements and is based at least on a comparison between this ratio and a threshold.

Optionally, the first frequency is between 0 and 50 Hz, the second frequency is between 50 and 150 Hz, and the threshold is between 50 and 100%.

Optionally, the first frequency is less than 50 Hz, the different frequencies comprise a family of frequencies less than 200 Hz and integer multiples of the first frequency, and the determination of the muscular fatigue is based on a calculation of a discrete integral of a function associating with each frequency of the family a force measurement taken at the level of the support element for supporting the lower limb in response to the electro-stimulation at that frequency.

The use of the verb “comprise” and its variants, as well as its conjugations in this document, cannot in any way exclude the presence of elements other than those mentioned. The use in this document of the indefinite article “a”, “an”, or the definite article “the” to introduce an element does not exclude the presence of a plurality of these elements.

The terms “first”, “second”, etc. are used in the context of this document exclusively to differentiate between different elements, without implying any order between these elements.

All of the preferred embodiments as well as all of the advantages of the device according to each example of the invention apply mutatis mutandis to the other examples of the invention and to the present method.

### BRIEF DESCRIPTION OF THE FIGURES

Further characteristics and advantages of the present invention will become apparent from the following detailed description, for the understanding of which reference is made to the attached figures which show:

FIG. 1, a perspective view of an example of the device according to an alternative embodiment of the invention;

FIG. 2, a side view of the device in FIG. 1;

FIG. 3, a perspective view of an example of a support element of the device and a measurement instrument;

FIG. 4, a perspective view of a preferred embodiment of the device according to the invention;

FIG. 5, a perspective view of an example of measurement instrument.

The drawings in the figures are not to scale. Similar elements are generally denoted by similar references in the figures. In the scope of this document, the same or similar elements may have the same references. Furthermore, the presence of reference numbers or letters in the drawings cannot be considered as limiting, even when these numbers or letters are indicated in the claims.

### DETAILED DESCRIPTION OF EMBODIMENTS OF THE INVENTION

The invention relates to a device for measuring a muscular force of a lower limb of a patient comprising a sitting for receiving the patient in a seated position and adapted to be placed on a substantially horizontal support as well as a support element for supporting the lower limb in order to receive at least a portion of the lower limb of the patient, the element being mechanically coupled to the sitting. The device also comprises a measurement instrument for measuring a force exerted by the lower limb at the level of the support element for supporting the lower limb. The measurement device is configured so that it remains substantially immobile relative to the support when a force is applied by the lower limb of the patient at the level of the support element for supporting the lower limb, due to the weight of the patient being exerted at the level of the sitting.

The device is designed to be used without an operator and is structurally stable and rigid so that the measurements are accurate and reproducible. The device is simple and light. The sitting can be placed on any support such as a table or a chair. The device is therefore very easily transportable and allows accurate measurements to be made without the need for additional equipment; the device is light, portable and can be taken easily to any location where there are patients on whom the muscular force measurements are to be made. The device moves where the patients are, so they don't have to go to a particular place. The accuracy of the measurements allows an appropriate management of the patients.

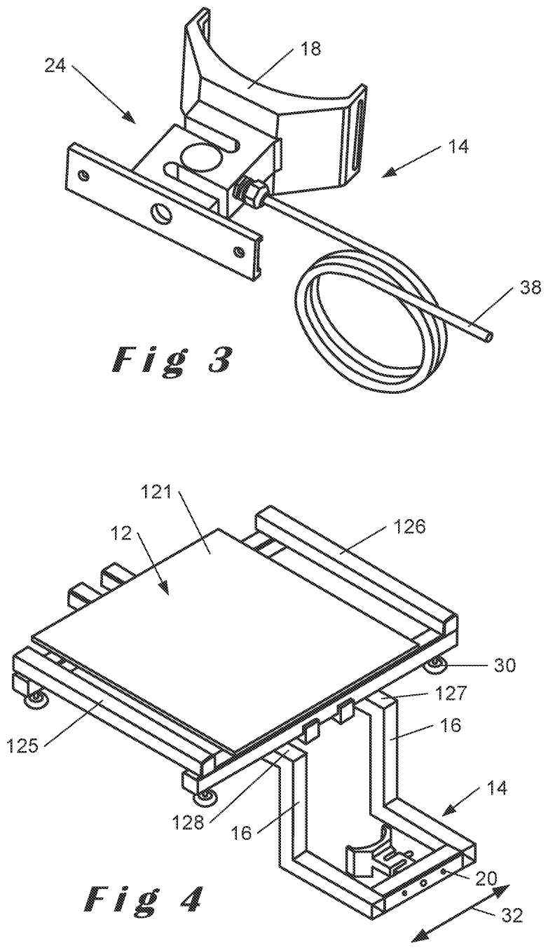

Preferably, the instrument is attached to a part arranged to be aligned with the direction of the force exerted by the lower limb at the level of the support element for supporting the lower limb. This position of the measurement instrument contributes advantageously to the accuracy of the measurements. It registers the force exerted by the lower limb at the level of the support element particularly directly and accurately, as it is located in the force line, typically near the support element. It is therefore not an intermediate force representative of this force that is measured at another position, but the exact force exerted by the lower limb at the exact level of the support element. This is an advantageous characteristic as the object of the present invention is to provide a device for measuring a muscular force providing accurate measurements. A preferred limitation of this characteristic is that the measurement instrument is a single part with the support element (visible in FIG. 3 hereafter commented).

FIG. 1 shows a perspective view of a device 10 according to an embodiment of the invention from the point of view of the positioning of the measurement instrument as explained below.

The device 10 allows perfectly to measure a muscular force of a lower limb of a patient. For example, it may be a muscle of the thigh, particularly the quadriceps or the hamstrings. It comprises a sitting 12 to receive the patient in a seated position, which helps to obtain accurate measurements of the muscular force of the muscle of the lower limb. The sitting 12 is suitable for placing on a substantially horizontal support; this may be a table or a chair. The sitting 12 has no floor support, which makes the device simple and light to transport. In contrast to the devices of the prior art, the fact that the device does not comprise a foot to hold it down to the floor makes it lighter in structure and portable. In other words, the sitting 12 is removable from the support and can be placed on any other essentially horizontal surface—measurements do not have to be taken in a dedicated location.

The sitting 12 is for example a tray 121 for receiving the patient, possibly padded for patient comfort. The surface of the sitting 12 is such that the entirety of the thigh of the patient rests on the sitting and the back of his knee is in contact with an edge of the sitting 12. The edge is sufficiently rounded to match the back of the knee and provide a comfort for the patient. The tray 121 measures approximately 40-70 cm by side—preferably 50-60 cm by side to have a balance between the overall dimension of the device 10 and the comfort of the patient in the seated position. The sitting 12 may further comprise a chassis 122 to which the tray 121 is attached. The chassis 122 provides rigidity to the sitting 12. The chassis 122 is, for example, a framework formed by two first bars 122, 123 supporting the tray 121 on its lower face and two other bars 124, 125 transverse to the first bars 122, 123 ensuring the rigidity of the assembly.

The device 10 further comprises a support element 14 for supporting the lower limb in order to receive at least a portion of the lower limb of the patient, in particular the leg (portion of the lower limb between the knee and the ankle), the ankle or the foot. The element 14 is mechanically coupled to the sitting 12. The support element 14 for supporting the leg, the ankle or the foot allows the lower limb to be held in place during measurement, thereby allowing to provide accurate measurements. The lower limb is immobilised in the element 14 to prevent a relative displacement between the element 14 and the lower limb as occurs in the prior art devices when the lower limb is simply resting against a cushion.

The element 14 can receive a front portion of the lower limb, below the knee—for example at the level of the tibia. The support element 14 for supporting the lower limb is for example a bight 18. The bight 18 comprises a rounded portion that match the curvature of the portion of the lower limb caught in the element 14 while laterally immobilising the lower limb. The bight 18 can be held by an armature 20 allowing to stiffen the area where the device is solicited by the lower limb of the patient. FIG. 3 shows another example of the element 14. The lower limb rests against the rounded portion of the bight 18. In these embodiments, a strap may be provided to further immobilise the lower limb against the support element 14. A wire 38 transfers the measurement signals towards a measurement processing unit.

The element 14 is mechanically coupled to the sitting 12 for example by at least one arm 16. The arm or the arms 16 extend out of the plane of the sitting 12. When the sitting 12 is placed on a substantially horizontal support, the arm or the arms 16 offset the support element 14 for supporting the lower limb below the plane of the sitting 12. When the patient is in a seated position, the element 14 is offset below the knee. Depending on the length of the arm or the arms 16, the support element 14 for supporting the lower limb may be at the level of the ankle, the tibia or the foot. According to FIG. 1, a single arm 16 mechanically couples the element 14 to the sitting 12; this makes the device lighter. According to FIG. 4, a pair of arms 16 mechanically couple the element 14 to the sitting 12, on either side of the element 14; this ensures more stability to the element 14. The arm or the arms 16 are orthogonal to the plane of the sitting 12, but as will be described later, the angle between the arm or the arms 16 and the sitting 12 may be different.

The arm or the arms 16 mechanically connect the element 14 to the sitting 12 through the chassis 122. The chassis 122 may comprise bars 127, 128 to which the arm or the arms 16 are attached. The arm or the arms 16 are attached to the bars 127, 128 along an axis 23.

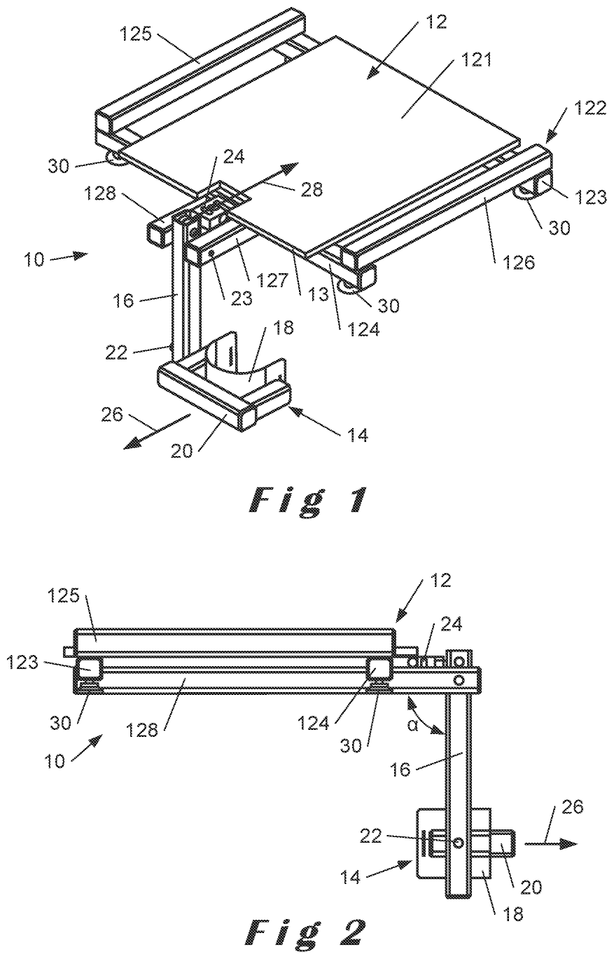

An example of the mechanical coupling of the support element 14 for supporting the lower limb to the sitting 12 is best seen in FIG. 2 which shows a side view of the device 10. The arm 16 offset the element 14 out of the plane of the sitting 12, underneath the latter when the device 10 is in the position of use on a substantially horizontal support. The bars 127, 128 are positioned under the tray 121 and shift the arm 16 beyond the tray 121 towards the front of the sitting 12. This allows the patient to sit on the sitting 12 with the back of the knee against a front edge 13 of the tray 121 and to have the support element 14 for supporting the lower limb facing a portion of his lower limb below the knee. The bars 127, 128 are attached to the bars 123, 124, parallel to the bars 125, 126. The bars 127, 128 extend towards the front of the device 10, projecting from a front edge 13 of the device. The element 14 can be attached to the arm 16 by an axle 22. The axle 22 secures the armature 20 to the arm 16.

FIG. 4 shows a perspective view of another example of embodiment of the device 10. In this example, the device comprises two arms 16 mechanically coupling the element 14 to the sitting 12—the rest of the device being the same as in FIGS. 1 and 2. The arms 16 offset the element 14 out of the plane of the sitting 12, underneath the latter when the device 10 is in use on a substantially horizontal support. Each arm 16 is connected to one of the bars 127, 128. The bars 127, 128 are positioned under the tray 121 and shift the arms 16 beyond the tray 121 towards the front of the sitting 12. This allows the patient to sit on the sitting 12 with the back of the knee against a front edge 13 of the tray 121 and to have the support element 14 for supporting the lower limb facing a portion of his lower limb below the knee. The bars 127, 128 are attached to the bars 123, 124, parallel to the bars 125, 126. The bars 127, 128 extend towards the front of the device 10, projecting from a front edge 13 of the device. In this example, the element 14 is mechanically coupled to the sitting by an ‘L’ (or ‘S’ or ‘Z’ depending on FIG. 4) frame formed by the bars 127, 128, the arms 16 and the armature 20. The element 14 may be attached to the arms 16 by an axle 22 not visible in FIG. 4. The axle 22 secures the armature 20 to the arms 16.

The support element 14 can be coupled by the single arm 16 or by two arms 16 forming the frame described in FIG. 4, but positioned under the sitting 12 and not beyond it as seen in the figures. The device 10 is then placed on a support with a clearance from below. The patient then exerts a force along a direction opposite to that of the arrow 26 in FIGS. 1 and 2.

Preferably, the device comprises only one support element 14 for supporting the lower limb, adjustable on the arm 16 to accommodate different lower limbs. In order to further simplify the structure of the device, and to facilitate its transport, the element 14 can be used for either lower limb. The element 14 can be removed and placed on either side of the arm 16 in FIGS. 1 and 2, facing the lower limb whose muscular force is to be measured. The axle 22 is for example a screw that removably holds the element 14 in place on the arm. It is also possible to adjust the position of the element 14 in height along the arm 16 to adapt to the size of the lower limbs of the patient.

The device 10 further comprises a measurement instrument 24 for measuring a force exerted by the lower limb at the level of the support element 14 for supporting the lower limb. The measurement instrument 24 may be a strain gauge or a mechanical dynamometer. The measurement instrument 24 can work in traction or in compression. According to various embodiments of the invention, the measurement instrument 24 may be positioned at numerous locations on the device 10, as long as the effort of the lower limb at the level of the support element 14 for supporting the lower limb is transferred to the measurement instrument 24. For example, the measurement instrument 24 could be located in the arm or the arms 16 to measure the deformation of the arms 16. As can be seen in FIGS. 1 and 2, the measurement instrument 24 could also be at the level of the sitting 12, between the bars 127, 128, in cooperation with the arm 16: the solicitation of the support element 14 by the lower limb would then propagate into the arm 16 which in turn would solicitate the measurement instrument 24.

According to FIG. 3, in an embodiment of the invention, the measurement instrument 24 and the support element 14 are one and the same part. In this configuration, the measurement instrument 24 registers the exact force exerted by the lower limb as it is located as close to it as possible. An S-shaped structure is deformed by solicitation of the lower limb. The measurement instrument 24 works in compression.

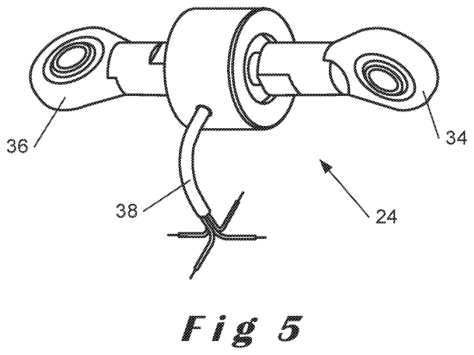

FIG. 5 shows a perspective view of another example of a measurement instrument 24. In this embodiment of the invention, the measurement instrument 24 is placed behind the lower limb, typically in the force line. The measurement instrument 24 therefore works in traction. It is articulated on two ball joints 34, 36 in order to accompany the movement of the lower limb in any direction of the latter. In FIGS. 1, 2, 4, the element 14, which rests on the front of the lower limb, is connected to the ball joint 34 or 36 of the measurement instrument 24, which is located behind the lower limb, the other ball joint 34, 36 being connected to an additional armature of the type of the armature 20. A wire 38 transfers the measurement signals towards a measurement processing unit.

The measurement instrument 24 may be coupled to the single arm 16 of FIGS. 1 and 2 or to both arms 16 of FIG. 4, being positioned under the sitting 12 and not beyond it. The sitting 12 is then placed on a support with a clearance from below.

In a normal use configuration, the device 10 is configured such that the force exerted by the lower limb at the level of the support element 14 for supporting the lower limb is substantially parallel to the sitting 12. In FIGS. 1 and 2, the arrow 26 shows the direction of application of a force by the lower limb of the patient at the level of the support element 14 for supporting the lower limb. Due to the weight of the patient exerted at the level of the sitting 12, the measurement device 10 remains essentially immobile in relation to the support on which the sitting is placed when this force is applied. In the configuration of FIGS. 1 and 2, in which the arm 16 extends orthogonally to the sitting 12, the arrow 26 is in a plane orthogonal to the sitting 12 comprising the arm 16 and, in this same plane, an arrow 28 shows the force that is applied in reaction to the instrument 24 positioned under the sitting 12.

According to the invention, the instrument 24 is attached to a part aligned with the direction of the force exerted by the lower limb, typically as a result of a contraction of the thigh. The immobility of the device ensured by the weight of the patient not only allows for ease of measurement but also for accuracy of measurement.

The support element 14 and the measurement instrument 24 can each be positioned relative to the lower limb in various combinations. The element 14 may be resting on the lower limb from in front or behind the limb; the measurement instrument 24 may also be in front or behind the limb and work in traction or compression. The device therefore offers a variety of structures allowing to adapt to different measurement needs. Preferably, along the force line, the following elements are arranged and in contact with each other in one of the following orders, depending on the orientation of the force:
measurement instrument, (portion of) lower limb, support element;
(portion of) the lower limb, support element, measurement instrument.

The device 10 may comprise members 30 for removably positioning the sitting on the support. These may be height adjustable members 30 so as to ensure the stability of the device on the support. In addition, the members 30 may be suction cups or clamps to hold the device in a same position on the support.

The arm or the arms 16 may be articulated with respect to the sitting 12, for example about the axis 23. As can be seen in FIG. 2 (but applicable to FIG. 4 as well), the angle α between the arm 16 and the sitting 12 may be adjustable, in a use configuration of the device. Once the desired angle α is achieved, the arm 16 is immobilised relative to the sitting 12 for the measurement. FIGS. 1 and 2 show an angle α of 90°; it can vary between 30° (position in which the arm 16 extends under the sitting) and 180° (position in which the arm 16 extends in the plane of the sitting 12, in front of it). The variation of the angle α is useful for taking measurements in various positions of the lower limb. The arm 16 can also be folded against the sitting 12 in a transport configuration of the device 10—this further facilitates the transport of the device 10. The angle α is then close to 0°.

In the embodiment shown in FIG. 4, the patient is positioned to the left or right of the sitting 12 to test the right or left lower limb. In FIG. 4, the arms 16 can also be movable relative to the sitting 12, in particular in translation laterally relative to the patient, according to the double arrow 32. The arms 16 can be positioned further to the left or right of the sitting 12 depending on whether the left or right lower limb is to be tested. In FIGS. 1 and 2, it is also possible for the patient to move to the left or right of the sitting 12 to test the right or left lower limb; the lateral translational mobility of the arm 16 according to the double arrow 32 is also applicable in the example of FIGS. 1 and 2.

The operation of the device will now be described in relation to the description of a method for measuring a muscular force of a lower limb of a patient. The method comprises a step of providing the device 10 as described above. The sitting 12 is placed on an essentially horizontal support; this may be a table or a chair. The patient then sits on the sitting 12, looking towards the front of the device, and his lower limb, the force of which is to be measured, is positioned in the support element 14 for supporting the lower limb. The force measurements are then taken by the measurement instrument 24 at the level of the element 14. To do this, the patient repeatedly contracts and releases his thigh, which tends to cause a movement of the bottom of the lower limb, under the knee, towards the front of the patient, thus soliciting the element 14. The instrument 24 takes measurements which are sent towards a measurement processing unit. The method is simple because the device 10 can be transported to any location and placed on any substantially horizontal support; the measurements are accurate because the measurement device remains substantially immobile relative to the support when the force is applied by the lower limb at the level of the support element for supporting the lower limb, due to the weight of the patient being exerted at the level of the sitting 12.

The patient is positioned on the sitting 12 so that his foot is off the ground. The support is therefore chosen so that the height between the surface of the sitting 12 on this support and the floor is greater than the length of the lower limb of the patient below his knee. This allows to avoid to distort the measurements due to the foot rubbing on the floor. According to FIG. 2, the patient is positioned so that the arm 16 is between his lower limbs; according to FIG. 4, one of the arms 16 is between his lower limbs. The entirety of his thigh rests on the sitting 12 and the back of his knee is in contact with the front edge 13 of the sitting 12. The front edge 13 of the sitting 12 is a support point for the exercise of the force by the lower limb as a result of the contraction of the thigh; the contraction of the thigh causes the rotation of the bottom of the lower limb towards the front of the device and of the patient, around the knee resting on the front edge 13. It is therefore not necessary to have an additional specific support point for the measurements. Such a position allows the reproducibility of the measurements as there is no adjustment to be made to the sitting 12 or to the support. In addition, to ensure the accuracy of the measurements, the patient sits in a straight-backed position. Thus, the seated position of the patient ensures the angulation. The thigh resting on the sitting 12 is parallel to the horizontal and the trunk vertical; the angle of the thigh and the bottom of the lower limb is guaranteed.

The method may comprise a step of positioning the support element 14 for supporting the lower limb opposite the lower limb whose muscular force is to be measured. In the configuration of FIGS. 1 and 2, the same element 14 can be used for both lower limbs of the patient, so the element 14 is mounted on the side of the arm 16 facing the lower limb to be tested and the height of the element 14 along the arm 16 is adjusted to the size of the lower limb of the patient. In FIG. 4, the patient sits on the sitting 12 so that the lower limb to be tested is positioned in front of the support element 14. In addition, in FIGS. 1, 2, 4, the support element 14 can be positioned opposite the lower limb whose muscular force is to be measured by lateral translation of the arm or the arms.

Depending on the type of measurements to be made, it is possible to adjust the angle α between the sitting 12 and the arm or the arms 16 mechanically coupling the support element 14 for supporting the lower limb to the sitting 12. Also, as described above, the support element 14 may be in front of or behind the lower limb, with the patient soliciting the element towards the front or the back. This allows other types of measurements to be taken.

The method allows to measure the fatigue of a muscle of a lower limb, such as the quadriceps or the hamstrings. The device ensures the measurement of the assembly of the quadriceps or the hamstrings.

The method may also comprise an electro-stimulation step generating a force exerted by the lower limb at the level of the support element 14 for supporting the lower limb, as described above.

More specifically, the method preferably comprises the following steps:
electro-stimulating a muscle of the lower limb at different frequencies,
taking force measurements of the lower limb at the level of the support so element 14 for supporting the lower limb in response to the aforementioned electro-stimulations,
determining a muscular fatigue on the basis of the force measurements of the lower limb taken in response to the above-mentioned electro-stimulations.

Thus, due to the device 10 and to the method according to this preferred embodiment, it is possible to determine the muscular fatigue of the muscle in a simple, safe and reliable way. In fact, the electro-stimulation use allows the muscle to be stimulated regardless of its fatigue and to make the muscle develop an involuntary force in response to the electro-stimulation. This step can then be performed at any time, even after a sports training, without putting the subject at risk of injury, regardless of the wishes of the subject. With a reduced number of electro-stimulations and an adapted frequency, this method does not induce additional muscular fatigue, and therefore does not distort the determination of the possible pre-existing muscular fatigue. In particular, the muscular fatigue before and after an execution of the determination method is essentially the same. This method allows to determine the muscular fatigue because it non-uniformly distorts the curve of the force developed by the muscle, and therefore the lower limb, in response to an electro-stimulation at a frequency dependent on that frequency. It is therefore possible to determine the muscular fatigue on the basis of the force measurements taken at different frequencies, for example by comparing these force measurements. This has the advantage of being independent of the context in which the method is executed. In particular, no comparison with such a known standard resting curve for the patient, no prior measurement and no specific execution conditions are required. Preferably the frequencies are between 0 and 1 kHz, preferably smaller than 500 Hz, more preferably smaller than 200 Hz. The frequencies preferably comprise a first and a second frequencies, p and p′, differing from each other by at least 10%. In this case, the force measurements comprise a first force measurement F1 of the lower limb at the level of the support element 14 for supporting the lower limb in response to the electro-stimulation of the muscle at the first frequency, and a second force measurement F2 of the lower limb at the level of the support element 14 for supporting the lower limb in response to the electro-stimulation of the muscle at the second frequency. The determination of the muscular fatigue then preferably comprises a calculation of an F1/F2 ratio and is based at least on a comparison between this ratio and a threshold. This threshold can be of the form F(μ)/F(μ′) where F is a patient-independent function expressing the force developed by an unfatigued muscle in response to an electro-stimulation of that muscle at a frequency, as a function thereof. For example, p is between 0 and 50 Hz, preferably between 10 and 40 Hz, more preferably about 20 Hz; p′ is between and 150 Hz, preferably between 90 Hz and 120 Hz, more preferably about 100 Hz; and the threshold is between 50 and 100%, preferably between 70 and 90%, more preferably it is about 80% when p is about 20 Hz and p′ is about 100 Hz. This has the advantage of being simple and allowing a quick and uncomplicated calculation to determine the muscular fatigue. It is also very effective. Indeed, as p differs by at least 10% from the second frequency, the ratio is fully affected by the non-uniformity and non-linearity of the deformation of the curve as a function of the muscular fatigue. In particular, F2 corresponds fairly roughly to F(μ′) for example for μ′=100 Hz (or greater), whereas F1 is the further away from F(μ) that it is affected by the fatigue of the muscle, for a frequency μ sufficiently smaller than μ′, for example between 10 and 30 Hz. Therefore, when the comparison of the F1/F2 ratio with the threshold allows to identify a difference, this difference expresses a muscular fatigue which can be determined implicitly and/or explicitly.

Preferably, μ is less than 50 Hz, and the frequencies comprise a family of frequencies less than 200 Hz being integer multiples of the first frequency, and preferably all such frequencies. The determination of muscular fatigue can then be based on a calculation of a discrete integral of a function associating with each frequency of the family a force measurement taken at the level of the support element 14 in response to the electro-stimulation at that frequency. This discrete integral typically corresponds to a Riemann sum. Preferably, p is less than 20 Hz, preferably less than 10 Hz, for a good calculation accuracy. Preferably, a comparison of the calculated discrete integral is performed with an expected area value, and the determination of the muscular fatigue is based on this comparison. The area value can be the area under the graph of the above-mentioned function F. As is well known in discrete calculation, the comparison then allows to evaluate a difference between this theoretical area for a non-fatigued muscle and its approximations by a Riemann sum for the muscle under consideration, and to determine the muscular fatigue on this basis accurately due to the number and the overall uniform distribution of the frequencies in the family.

In general, the muscular fatigue can be determined by calculation or calculations on the force measurements taken (e.g. by ratio of two forces, by discrete integral calculation, as mentioned above, and/or by a combination of these techniques) and/or comparison of at least one such calculation to at least one expected value.

The present invention has been described above in connection with specific embodiments, which are illustrative and should not be considered limiting.

In general, it will be apparent to a person skilled in the art that the present invention is not limited to the examples illustrated and/or described above.

1. A device for measuring a muscular force of a lower limb of a patient, the device comprising:
a sitting configured to receive the patient in a seated position and to be placed on a horizontal support,
a support element mechanically coupled to the sitting, and configured to support the lower limb and to receive at least a portion of the lower limb of the patient, and
a measurement instrument configured to measure a force exerted by the lower limb at a level of the support element for supporting the lower limb,
the instrument being attached to a part arranged to be aligned with the direction of that force,
the measurement device being configured such that it remains immobile relative to the support when a force is applied by the lower limb of the patient at the level of the support element for supporting the lower limb, due to the weight of the patient being exerted at the level of the sitting.

2. The device according to claim 1, wherein the sitting is a tray extending continuously by at least 40 centimeters along two perpendicular axes.

3. The device according to claim 1, further comprising at least one arm mechanically coupling the support element for supporting the lower limb to the sitting, and offsetting the support element out of the plane of the sitting, under the sitting, in a use configuration of the device.

4. The device of claim 3, further comprising a single support element configured to support the lower limb, adjustable on the arm or the arms to adapt to different lower limbs.

5. The device according to claim 3, wherein the arm or the arms are movable in translation laterally with respect to the sitting.

6. The device according to claim 1, wherein the measurement instrument is a single part with the support element.

7. The device according to claim 1, wherein the measurement instrument is a strain gauge or a mechanical dynamometer.

8. The device according to claim 1, wherein the measurement instrument is adapted to work in compression or in traction.

9. The device according to claim 1, further comprising members configured to removably position the sitting on the support.

10. The device according to claim 1, wherein the force exerted by the lower limb at the level of the support element for supporting the lower limb is parallel to the sitting, in a use configuration of the device.

11. The device according to claim 1, configured such that the support element for supporting the lower limb is adapted to receive a portion of the lower limb below a knee of the patient, wherein the support element is a bight conformed to adapt to the portion of the lower limb resting against the support element.

12. A method for measuring a muscular force of a lower limb of a patient, comprising the following steps:
providing the device according to claim 1,
placing the sitting on a substantially horizontal support,
positioning the patient seated on the sitting,
positioning a lower limb of the patient whose force is to be measured in the support element for supporting the lower limb, and
taking force measurements of the lower limb at the level of the support element for supporting the lower limb.

13. The method according to claim 12, wherein the entirety of a thigh of the patient rests on the sitting, wherein the back of an associated knee is in contact with an edge of the sitting and the positioning of the patient is such that an associated foot is off the ground.

14. The method according to claim 12, wherein the support is a table.

15. The method according to claim 12, further comprising an electro-stimulation step generating a force exerted by the lower limb at the level of the support element for supporting the lower limb.

16. The method according to claim 12, further comprising the following steps:
electro-stimulating a muscle of the lower limb at different frequencies,
taking force measurements of the lower limb at the level of the support element for supporting the lower limb in response to the above-mentioned electro-stimulations, and
determining a muscular fatigue on the basis of the force measurements of the lower limb taken in response to the above-mentioned electro-stimulations.

17. The method according to claim 16, wherein the different frequencies comprise a first frequency and a second frequency, differing by at least 10% from each other, and the force measurements comprise a first force measurement of the lower limb at the level of the support element for supporting the lower limb in response to the electro-stimulation of the muscle at the first frequency, and a second force measurement of the lower limb at the level of the support element for supporting the lower limb in response to the electro-stimulation of the muscle at the second frequency.

18. The method of claim 17, wherein the step of determining a muscular fatigue comprises a calculation of ratio between the first and the second force measurements and is based at least on a comparison of the ratio with a threshold.

19. The method of claim 18, wherein the first frequency is between 0 and 50 Hz, the second frequency is between 50 and 150 Hz, and the threshold is between 50 and 100%.

20. The method according to claim 17, wherein the first frequency is lower than 50 Hz, wherein the different frequencies comprise a family of frequencies lower than 200 Hz and integer multiples of the first frequency, and wherein the determination of the muscular fatigue is based on a calculation of a discrete integral of a function associating with each frequency of the family a force measurement taken at the level of the support element for supporting the lower limb in response to the electro-stimulation at that frequency.
